(19) United States
(12) Patent Application Publication
(10) Pub. No.: US 2018/0322957 A1
(43) Pub. Date: Nov. 8, 2018

(54) DEVICES AND METHODS FOR MANAGING RISK PROFILES

(71) Applicants: David A. DILL, Newtown, GA (US); Cheryl Dill, Newtown, PA (US); Stephanie C. Dill, New York, NY (US); Andrea P. Terril, Hastings-on-Hudson, NY (US)

(72) Inventors: David A. DILL, Newtown, GA (US); Cheryl Dill, Newtown, PA (US); Stephanie C. Dill, New York, NY (US); Andrea P. Terril, Hastings-on-Hudson, NY (US)

(21) Appl. No.: 16/031,908

(22) Filed: Jul. 10, 2018

Related U.S. Application Data
(63) Continuation of application No. 14/873,889, filed on Oct. 2, 2015.
(60) Provisional application No. 62/060,940, filed on Oct. 7, 2014.

(51) Int. Cl. G16H 50/30; G06Q 40/06; G16H 10/40; G16H 10/60

(52) U.S. Cl. CPC G16H 50/30; G16H 10/60; G16H 10/40; G06Q 40/06

(57) ABSTRACT

The present application provides a method for determining and managing a subject's risk profile. The risk profile may include the presence or absence of nocturia. The present application further provides methods of using such a risk profile, such methods for underwriting or managing the cost of life insurances or health insurances, methods for calculating risk associated with the risk profile in an insurance candidate/insured subject, and methods for improving lifespan of the insured based on the risk profile.

This application is a Continuation of U.S. application Ser. No. 14/873,889, filed Oct. 2, 2015, which claims the benefit of U.S. Provisional Application Ser. No. 62/060,940, filed Oct. 7, 2014. The entirety of the aforementioned application is incorporated herein by reference.

### FIELD

This application relates generally to devices and methods for the creation, management and utilization of risk profiles that can be used by insurance companies in the underwriting and cost control of insurance policies. The risk profiles can also be used in the selection of candidates for clinical trials.

### BACKGROUND

Underwriting is the process an insurance company uses to determine whether or not a potential customer is eligible for insurance, and the rate that the potential customer should pay for the insurance if eligible. The purpose of insurance underwriting is to spread risk among a pool of insured in a manner that is both fair to the customer and profitable for the insurer. In the area of life insurance and medical insurance, an insurance company typically looks at a number of factors during the underwriting process in order to evaluate a potential customer in terms of risk. These factors enable the insurer to decide whether or not the potential customer is insurable. If the potential customer is insurable, these factors help place them into an appropriate risk group. Some of the factors considered are age, sex, current health/physical condition, personal health history, family health history, financial condition, personal habits/character, occupation, and hobbies. With the ever increasing cost of medical care, there exist a need to develop devices and methods to manage the risk profiles of insured and reduce the cost of insurance.

### SUMMARY

One aspect of the present application relates to a computer system for managing the risk and the cost of insurance for a subject. The computer comprises a processor and a memory device, wherein the memory device stores a risk point matrix and wherein the processor is configured to: (1) receive or generating a risk profile concerning the subject, wherein the risk profile comprises a plurality of risk factors including the severity level of the subject's nocturia; (2) assign a risk point value to the subject based on the severity level of the subject's nocturia and a risk point matrix stored in the memory device; (3) assign additional risk point values to the subject based on other risk factors in the subject's risk profile; (4) determine a total risk point value of the subject; and (5a) if the total risk point value equals to or exceeds a predetermined threshold value, reject the subject as an insurance candidate, or (5b) if the total risk point value is below the predetermined threshold, generate an insurance premium for the subject based on an insurance premium matrix stored in the memory device. The insurance premium matrix may be modified from time to time.

In some embodiments, the presence and/or severity of nocturia is determined by measuring the level of urinary prostaglandin E2 (PGE2), urinary prostaglandin F2α (PGF2α), urinary nerve growth factor (UNGF), plasma arginine vasopressin (AVP), plasma atrial natriuretic hormone (ANH), plasma renin-angiotensin aldosterone (RAA) or combinations thereof. In other embodiments, nocturia is screened by including a question about the average frequency of urination at night in an insurance application form or insurance survey form, or in a form to be filled out by the applicant's primary care physician or urologist.

In some embodiments, the method further comprises the step of providing insured people with information about behavior, diet, medical care or household safety that would help to improve health, happiness and/or longevity. In some embodiments, the information is provided electronically on a regular basis. In some embodiments, information about behavior or diet or medical care or household safety (among other strategies) is provided to people with nocturia symptoms. In some embodiments, the method further comprises the step of providing insured people having nocturia symptoms with information about behavior and/or diet that would help to reduce severity of nocturia or to reduce the risk associated with nocturia, e.g., strategies to reduce the risk of a fall during the trip to the bathroom at night.

In some embodiments, the method further includes a step of providing insured people with devices, such as pedometers, that would help to encourage the recipient to do physical exercises on a regular basis. In some embodiments, the devices are provide to those who have symptoms of nocturia.

In some embodiments, the method further includes a step of providing insured people with financial incentives, such as a reduced annual insurance premium, rebates or coupons or discounts to purchase certain items or services, based on their adoption of a healthier life style or as incentives to adopt a healthier life style. In some embodiments, the incentives are provided to those with proof of positive behavior (e.g., flu shots, vaccinations, annual physical, blood pressure tests, weight, blood or urine test results, regular physical exercise, etc.).

In some embodiments, the incentives are provided to insured people who have nocturia symptoms and have adopted a healthier life style.

Another aspect of the present application relates to a computer system for managing the cost of insurance for an insured subject. The computer system comprises a processor and a memory device, wherein the memory device stores a risk point matrix and wherein the processor is configured to: receive a risk profile concerning the subject, wherein the risk profile comprises a plurality of risk factors including the severity level of the subject's nocturia; assign a risk point value to the subject based on the severity level of the subject's nocturia and the risk point matrix stored in the memory device; assign additional risk point values to the subject based on other risk factors in the subject's risk profile; and retrieve information about positive behavior that reduces risks associated with nocturia and/or other risk factors from the memory device and electronically deliver the information to the subject or to the insurance company. The risk point matrix may be modified from time to time.

Another aspect of the present application relates to a computer system for selecting candidate for a clinical trial. The computer system comprises a processor and a memory device, wherein the memory device stores a selection point matrix and wherein the processor is configured to: (1) receive a selection profile concerning a potential candidate, wherein the selection profile comprises a plurality of selection factors including the severity level of the potential candidate's nocturia; (2) assign a selection point value to the subject based on the severity level of the potential candidate's nocturia and the risk point matrix stored in the memory device; (3) assign additional selection point values to the subject based on other selection factors in the potential candidate's selection profile; (4) determine a total selection point value of the potential candidate; and (5a) if the total candidate point value equals to or exceeds a predetermined threshold value, accept the potential candidate as a clinical trial candidate, or (5b) if the total risk point value is below the predetermined threshold, reject the potential candidate as a candidate for the clinical trial.

Another aspect of the present application relates to a tangible computer readable medium having instructions stored thereon for insurance underwriting based on risk factors including the severity of nocturia. The instructions when executed by a processor causing the processor to: (1) receive or generate a risk profile concerning the subject, wherein the risk profile comprises a plurality of risk factors including the severity level of the subject's nocturia; (2) assign a risk point value to the subject based on the severity level of the subject's nocturia and the risk point matrix stored in the memory device; (3) assign additional risk point values to the subject based on other risk factors in the subject's risk profile; (4) determine a total risk point value of the subject; and (5a) if the total risk point value equals to or exceeds a predetermined threshold value, reject the subject as an insurance candidate, or (5b) if the total risk point value is below the predetermined threshold, generate an insurance premium for the subject based on an insurance premium matrix stored in the memory device.

Another aspect of the present invention relates to a method for managing the cost of life insurances or health insurances. The method comprises the steps of: screening an insured subject for the presence or absence of one or more risk factors including nocturia, wherein the presence and severity of nocturia is determined by: (1) an answer provided by the insured subject or an authorized representative of insured subject or medical professional in response to a question about the average frequency of urination at night, and/or (2) measuring a level of urinary prostaglandin E2 (PGE2), urinary prostaglandin F2α (PGF2α), urinary nerve growth factor (UNGF), plasma arginine vasopressin (AVP), plasma atrial natriuretic hormone (ANH), plasma renin-angiotensin aldosterone (RAA) or combinations thereof in the insured subject; providing to the insured subject information about how to reduce risk associated with one or more risk factors present in the insured subject, wherein the information includes information about behavior, or medications, or diet, or exercises that would help to reduce severity of nocturia or to reduce the risks associated with nocturia; and providing incentives to the insured subject to encourage positive behavior by the insured subject, wherein the incentives include reduced insurance premium or discounts or other form of financial or other reward based on proven performance of one or more positive behavior.

### DETAILED DESCRIPTION

Those who suffer from nocturia in a significant way tend to have much higher death rates than those who have no nocturia symptoms. For example, in a published study covering data for 16,000 people over 12 years, among women ages 65-90, the 12 year survival rate for those with no nocturia symptoms was 68%. The 12 year survival rate for those who had 3 or more nocturia events per night was 22% (Kupelion et al., The Journal of Urology, 2011, 185:571-577). Among men and other age groups, the data were also profoundly different depending on the level of nocturia severity.

Individuals with nocturia symptoms, however, are not properly assessed by insurance companies based on current underwriting methods. The result of the insurance carrier not identifying the degree of nocturia in insurance candidates is that insurance candidates with mild nocturia (e.g., 1-2 voids per night), moderate nocturia (e.g., 3-4 voids per night) or severe nocturia (e.g., >4 voids per night) are underpriced, and insurance candidates with no nocturia (e.g., no void per night) are over-priced. This mismatch not only poses a significant disadvantage for some insurance candidates by not being assigned the appropriate premium, but policyholders (for mutual companies) and shareholders (for stock companies) do not maximize value given their inability to effectively manage this risk.

Moreover, risks associated with nocturia (e.g., accidents caused by fatigue as a result of interrupted sleep, and falls in frequent night trips to the bathroom) are manageable and can be significantly reduced if an insurance policy holder with nocturia is well informed of the risk and takes the necessary action to avoid the risk. Furthermore, there are various strategies the nocturia sufferers could employ to reduce their symptoms.

### Methods for Determining and Managing the Risk Profile of a Subject

One aspect of the present application provides methods for determining and managing the risk profile of a subject. In some embodiments, the risk profile includes the presence or absence of nocturia, as well as the severity of nocturia if present. In some embodiments, the risk profile further includes the general biological information (e.g., sex, age, weight, height, ethnical background, vital sign measurements such as heart rate, blood pressure, blood type, body mass index, sleep pattern, etc.); smoking history, alcohol consumption, drug use history, personal history and family history of cardiovascular disease, personal history and family history of cancer, personal history and family history of immunological disorders, personal history and family history of mental disorders, personal history and family history of cardiovascular diseases; personal history on surgery; personal history and family history of chronic conditions such as high cholesterol (cholesterolemia), high blood pressure (hypertension), and depression, etc. In some embodiments, the risk profile of the subject further includes the subject's driving history, such as the history of driving accident and traffic violations, the cars the subject drives, the use of motor cycles, common driving routes, routine travel patterns, frequency of driving, etc.

In some embodiments, the risk profile of the subject further includes the subject's job history and history of dangerous pursuits such as racing, mountain climbing, diving and hang gliding. In some embodiments, the risk profile of the subject further includes the subject's work history, such as the job held, type of physical activities required on the job, average working hours per week, etc. In some embodiments, the risk profile of the subject further includes the subject's travel history to foreign countries. In some embodiments, the information included in a subject's risk profile is categorized into a number of risk factors and each risk factor is assigned one or more risk point values for the determination of a final risk point value for the subject.

The risk profile may be generated by collecting information provided by the subject, the subject's representative, the subject's physician, other medical professional and other insurance companies, such as auto insurance companies, with the subject's permission.

In some embodiments, personal health data of the subject may be collected from information generated by one or more self-monitoring health sensors or devices. In some embodiments, the health sensor or device includes a processor, a memory, user interfaces (e.g., a display screen, a touch screen, a keyboard, an analog control panel, etc.) and optionally a transmitter that may remotely connect to a computer and transmit collected data to the computer. A human operator (e.g., the subject) may also enter inputs to operate the health sensors (e.g., setting sensor parameters, storing and displaying data, etc.) via the user interfaces. Additionally or alternatively, the human operator may control the health sensors by entering inputs in a computing device (e.g., a smart phone, a tablet computer, a personal computer, etc.) connected to the health sensors either directly or remotely via the network. In some embodiments, the personal health sensor includes a device capable of detecting, collecting, storing, transmitting, and/or displaying data related to the personal health of the subject to whom the sensor is attached. The health sensor may be wearable, implantable, ingestible, hand-held, placed off the body of the subject or otherwise attached to the subject. The health sensor may also be capable of determining if the subject falls or is otherwise physically or mentally distressed or endangered.

In some embodiments, the health sensor are devices used to measure various physiological parameters of subject. Examples of such devices include, but are not limited to, heart rate monitors, pulse oximeters, blood pressure monitors and sleep pattern monitors. In some embodiments, the health sensors are devices used to measure various fitness activities carried out by the subject. Examples of such devices include, but are not limited to, pedometers, smart watches, electronic fitness bracelets, mobile phones with motion sensors. Devices that measure fitness activities may also include electronic devices attached to exercise or recreational equipment such as a bike. In some other embodiments, the health sensor is a medical device such as a blood biometric analyzer (e.g., a blood glucose meter) used to measure biometric marker levels in the blood such as cholesterol levels, blood glucose levels, nutrient levels, etc., or an electromyography device used to measure electrical activities in the muscles to analyze biomechanics. In some other embodiments, the health sensors is a medication monitoring device (e.g., smart pills) used to measure medication usage by the subject (e.g., correct intake of medicine, failure to intake medicine, refills, etc.). Additional examples of the health sensor may include temperature sensors, humidity sensors, etc., used to measure other factors such as environmental factors that may affect the health or wellness condition of the subject.

In some embodiments, data gathered by the health sensor is stored in a memory device. In some embodiments, the memory device is part of a computer system. In other embodiments, the data is temporarily stored in an individual memory device such as flash drive before being transmitted to the memory device in a computer system. In some embodiments, data gathered by the health sensors is transmitted directly to a computer system (including for example, a smart phone) via a network.

The health sensor may only collect and send the personal health data with the subject's (or his or her legal guardian's or caretaker's) full understanding and acceptance of published terms and conditions for collection and use of that data. In some embodiments, before the health sensor execute instructions to collect or process this data, a visual or other prompt at the health sensor may alert the subject to such action. The prompt allows the subject to “opt out” of some or all collection of the personal health data. The health data of the subject is eventually stored in the memory device in a computer system. A processor of the computer system may execute instructions stored in a memory of the computer system to retrieve data stored in the memory device and convert the data into a particular format (e.g., for efficient storage). In some embodiments, the computer converts the risk profile data into a risk point value based on a matrix that assigns a risk point value to each factor (or element) of the risk profile.

In some embodiments, initial incentives are offered to the subject for supplying data to his or her risk profile. In some embodiments, continued incentives are offered to encourage the subject to update his/her risk profile. Examples of the incentives include, but are not limited to, financial incentives such as a discount on the insurance premium.

The risk profile may be used in methods for underwriting life insurance or health insurance. The risk profile may also be used in managing the cost of life insurances and health insurances. The risk profile may also be used for other purposes, such the determination of candidacy for a clinical trial. The present application further provides a number of methods of using such a risk profile.

Another aspect of the present application provides a method for managing cost of insurances, such as life insurance, health insurance, disability insurance or long term care insurance. The method comprises the steps of: (1) screening an insurance candidate for the existence or absence of one or more risk factors; and (2) providing information and/or incentives to encourage positive behavior by insured people. In some embodiments, the one or more risk factors comprise the severity of nocturia. In some embodiments, the present application provides a method for selecting candidates for the clinical trial using the candidates' risk profiles.

As used herein, the term “nocturia” refers to the complaint that the individual frequently has to wake at night one or more times for voiding. The two primary causes of nocturia are hormone imbalances and vesical problems. Two major hormones that regulate the body's water level are arginine vasopressin (AVP) and atrial natriuretic hormone (ANH). AVP is an antidiuretic hormone produced in the hypothalamus and stored in and released from the posterior pituitary gland. AVP increases water absorption in the collecting duct systems of kidney nephrons, subsequently decreasing urine production. It is used to regulate hydration levels in the body. ANH, on the other hand, is released by cardiac muscle cells in response to high blood volume. When activated, ANH releases water, subsequently increasing urine production. Elevated levels of urinary nerve growth factor (UNGF), prostaglandin E2 (PGE2) and prostaglandin F2α (PGF2α) are also found in patients with overactive bladder and nucturia.

Nocturia has four major underlying causes: global polyuria, nocturnal polyuria, bladder storage disorders, or mixed etiology. The first two processes are due to irregular levels of AVP or ANH. The third process is a vesical problem.

Global polyuria is the continuous overproduction of urine which is not only limited to sleep hours. Global polyuria occurs in response to increased fluid intake and is defined as urine outputs of greater than 40 mL/kg/24 hours. The common causes of global polyuria are primary thirst disorders such as diabetes mellitus and diabetes insipidus (DI). DI is caused by irregular water levels in the body. Urination imbalance may lead to polydipsia or excessive thirst to prevent circulatory collapse. Central DI is caused by low levels of AVP that helps regulate water levels. In nephrogenic DI, the kidneys do not respond properly to the normal amount of AVP. Diagnosis of DI can be made by an overnight water deprivation test. This test requires the patient to eliminate fluid intake for a fixed period of time, usually around 8-12 hours. If the first morning void is not highly concentrated, the patient is diagnosed with DI. Central DI usually can be treated with desmopressin, a synthetic replacement of AVP. Although there is no substitute for nephrogenic DI, it may be treated with careful regulation of fluid intake.

Nocturnal polyuria is defined as an increase in urine production during the night but with a proportional decrease in daytime urine production that results in a normal 24-hour urine volume. With the 24-hour urine production within normal limits, nocturnal polyuria can be translated to having a nocturnal polyuria index (NPi) greater than 35% of the normal 24-hour urine volume. NPi is calculated simply by dividing NUV by the 24-hour urine volume. Similar to the inability of control urination, a disruption of arginine vasopressin (AVP) levels has been proposed for nocturia. Compared with the normal patients, nocturia patients have a nocturnal decrease in AVP level. Other causes of nocturnal polyuria include diseases such as congestive heart failure, nephritic syndrome and hepatic failure; or lifestyle patterns such as excessive nighttime drinking. The increased airway resistance that is associated with obstructive sleep apnea may also lead to nocturnal polyuria. Obstructive sleep apnea has been shown to cause increases in renal sodium and water excretion that are mediated by elevated plasma ANH levels.

Bladder storage disorders are defined as any factors that increase the frequency of small volume voids. These factors are usually related to lower urinary tract symptoms that affect the capacity of the bladder. Patients with nocturia who do not have either polyuria or nocturnal polyuria according to the above criteria, will most likely have a bladder storage disorder that reduces their nighttime voided volume or a sleep disorder. Nocturnal bladder capacity (NBC) is defined as the largest voided volume during the sleep period. Decreased NBC can be traced to a decreased maximum voided volume or decreased bladder storage. Decreased NBC can be related to other disorders such as prostatic obstruction, neurogenic bladder dysfunction, learned voiding dysfunction, anxiety disorders, or certain pharmacological agents.

A significant number of nocturia cases occur from a combination of etiologies. Mixed nocturia is more common than many realize and is a combination of nocturnal polyuria and decreased NBC. In a study of 194 nocturia patients, 7% were determined to have simple nocturnal polyuria, 57% had decreased NBC, and 36% had a mixed etiology of the two (Weiss J P et al., (1998) “Nocturia in adults: Etiology and classification.” Neurourology and Urodynamics 17: 467-72). The etiology of nocturia is multifactorial and often unrelated to an underlying urological condition. Mixed nocturia is diagnosed through the maintenance and analysis of bladder diaries of the patient. Assessment of etiology contributions are done through formulas.

In some embodiments, the presence of nocturia or the severity of nocturia is determined by measuring the level of PGE2 and/or PGF2a in the urine. Elevated levels of PGE2 and/or PGF2a in the urine greatly increase the likelihood of nocturia. For example, nocturia sufferers can have PGE2 levels in their urine that are up to 8 times normal levels or more and average levels for nocturia patients are between 4 and 5 times normal levels. The methods for determining urinary PGE2 or PGF2a levels are well known in the art.

In some embodiments, nocturia is screened by measuring the level of UNGF in the urine. Elevated levels of UNGF in the urine increase the likelihood of nocturia. The methods for determining UNGF levels are well known in the art. In some embodiments, the level of UNGF is determined by ELISA.

In other embodiments, nocturia is screened by measuring the level of AVP in the plasma or urine. Decreased levels of AVP in the plasma or urine increase the likelihood of nocturia. The methods for determining plasma AVP levels are well known in the art.

In other embodiments, nocturia is screened by measuring the level of ANH in the plasma or urine. Elevated levels of ANH in the plasma or urine increases the likelihood of nocturia. The methods for determining plasma ANH levels are well known in the art.

In other embodiments, nocturia is screened by measuring the level of renin-angiotensin aldosterone (RAA) in the plasma or urine. Decreased levels of RAA in the plasma or urine increases the likelihood of nocturia. The methods for determining plasma RAA or urine RAA levels are well known in the art.

As used herein, the term “elevated levels” refers to levels that are higher than a predetermined reference level, and the term “decreased levels” refers to levels that are lower than a predetermined reference level.

In some embodiments, nocturia is screened by including a question in the insurance application form or insurance survey form about the average frequency of urination at night.

In some embodiments, the severity of nocturia is determined based on one or more factors selected from the group consisting of urinary levels of PGE2, PGF2a and UNGF, plasma levels of AVP, ANH and RAA, as well as information provided in an insurance application or survey forms.

In some embodiments, the “severity of nocturia” is defined as follows:

no nocturia (no night void per night),

mild nocturia (1 night void per night), or

moderate nocturia (2 night voids per night),

severe nocturia (3 or more night voids per night).

The term “night void” is defined as a void that occurs after the subject goes to bed at night and before the subject gets up in the morning.

In other embodiments, the “severity of nocturia” is defined as follows:

no nocturia (no night void per night or less than 0.5 average night void per night),

mild nocturia (more than 0.5, but less than 1.5 average night void per night), or

moderate nocturia (more than 1.5, but less than 2.5 average night voids per night),

severe nocturia (more than 2.5 average night voids per night).

The term “average night void(s),” as used herein, refers to the average night void(s) per night over a two-week (14 days) period of time. For example, if a subject had a total of 12 night voids over a period of 14 days, the subject has an average night void of 0.86 and is thus categorized as having mild nocturia based on the criteria described above.

In some embodiments, the severity of nocturia is converted into a risk point value based on a predetermined conversion factor or a risk factor/risk point value matrix stored in a computer. For examples, no nocturia may be assigned a risk point value of zero. Mild nocturia may be assigned a risk point value of 3. Moderate nocturia may be assigned a risk point value of 6 and severe nocturia may be assigned a risk point value of 12. In some embodiments, a risk factor/risk point value matrix is created for each of the risk factors in the risk profiles.

In some embodiments, the method further comprises the step of assigning a risk point value to each risk factor in a subject's risk profile and determining a total risk point value for the subject. In some embodiments, the step of assigning a risk point value to each risk factor in a subject's risk profile is performed with a processor in a computer system using the risk factor/risk point value matrix stored in a memory device in the computer system. In some embodiments, the processor The risk factor/risk point value matrix provides one or more risk point values to each risk factor. The co-presence of certain risk factors may result in a risk point value that is greater than the summation of individual risk point values of the risk factors. The risk factor/risk point value matrix is revised periodically to reflect new findings and development that impact on the risk factors.

In some embodiments, the step of providing information and/or incentives to encourage positive behavior by insured people is performed by the processor in the computer system. The information and/or incentives are delivered electronically to the insured people. In some embodiments, the computer analyzes each insured person's risk profile and provides insured people having one or more risk factors with information about treatment for or strategies to manage such risk factors. In some embodiments, the information further includes information about doctors who provide such treatment in the local area of the insured.

In some embodiments, the method further comprises the step of providing insured people having nocturia symptoms with information about treatment for nocturia. In some embodiments, the information further includes information about doctors who provide such treatment in the local area of the insured. In some embodiments, the information further includes how to reduce risk associated with one or more risk factors present in the insured subject. The information may include information about behavior, medication, diet, drinking habit (e.g., drinking timing or types of fluid choices or other drinking strategies), and/or exercises that would help to reduce severity of the risk factor or to reduce the risks associated with the risk factor. In some embodiments, the information is provided electronically to the insured subject.

In some embodiments, the method further comprises the step of providing insured people having nocturia symptoms with information about behavior and/or diet that would help to reduce severity of nocturia or to reduce the risk associated with nocturia, e.g., fall during the trips to the bathroom at night. In some embodiments, the information is provided on a regular basis. Examples of behavior exercises include, but are not limited to, reducing caffeine and/or alcohol intake, wearing compression stockings through the day to prevent fluid from accumulating in the legs, elevating legs for a period of time prior to going to bed each night, causing less overnight urinary output, reducing consumption of any fluids in hours before bedtime, variations in diet, and increasing the amount of daily physical exercise. In some embodiments, the information is provided electronically to the insured subject.

In some related embodiments, the method further includes a step of providing incentives to encourage positive behavior and discourage negative behavior. Examples of positive behavior include, but are not limited to, active lifestyle, regular physical exercise, regular mental exercise, annual flu shots, updated vaccinations, annual physical, regular check on blood pressure and weight, regular blood and urine tests, regular physical exercise, etc. good sleeping habits, proper nutritional diet, taking medication on time, use of low dose aspirin, use of calcium supplements, use of vitamin D and C, use of dental floss, use of sunscreen, consumption of fruits and vegetables, avoidance of risky behavior, wearing seat belts, not driving under the influence of alcohol, and not driving when overly tired. Examples of negative behavior include, but are not limited to, lack of physical exercise, overeating, insufficient sleep, etc. The definition of positive and negative behavior may vary among insured subjects. For example, playing tennis is a positive behavior for an insured subject who is 30 years old, but could be a negative behavior for an insured subject who is 80 years old. In some embodiments, the method comprises the step of calculating an insurance premium adjustment based on the determined positive or negative behavior. In some embodiments, the insurance premium adjustment is calculated based on algorithms that analyze how behaviors affect risk, or based on an analysis of claims data without analyzing any corresponding personal health data.

The incentives may include financial incentives such as reduced insurance premiums or discounts for gym memberships and non-financial incentives such as free workout DVDs, free or discounted subscription to a weight loss program, free or discounted enrolment to programs to encourage quitting tobacco or alcohol use, free or discounted access to stress reduction and wellness strategies, mammograms, flu shots, and other strategies to improve wellness and life spans.

In some embodiments, the method further includes a step of providing insured subjects with devices, such as pedometers, that would help to encourage the recipient to do physical exercises on a regular basis, and/or devices that monitor the subject's health or stress levels and sends out an alert if evidence of increasing risk or need for medical care becomes apparent (such as a medical alert bracelet or other device that would sense a dangerous fall, or increased blood pressure or pulse or blood sugar level or other dangerous condition). In other embodiments, the method includes a step of providing insured subjects with free or discounted home safety devices such as smoke detectors and carbon monoxide detectors, as well as services for changing batteries in such devices.

In some embodiments, the method includes a step of providing an insured subject having nocturia symptoms with financial incentives, such as a reduced annual insurance premium, rebates or coupons to purchase certain items or services, based on their proven walking activities (e.g., walking at least 10,000 steps per day) that could be submitted online.

In some related embodiments, the method further includes a step of providing clear information to insured people about the life expectancy implications of their choices. For example, telling a person that his expected lifespan on average would be increased by 1 year for every 5 pounds that he lost or 2 years if he walked an extra 4000 steps per day, might convince some to improve their chances. The result would be healthier people, longer life spans, and higher profits for the insurance companies, whether they are, for example, life insurance or health insurance companies. As these strategies prove their value, such insurance companies could also advertise that they help their customers live longer, safer, and healthier lives than those of other insurance companies by motivating, encouraging, rewarding, and educating them.

In some related embodiments, the method further includes a step of encouraging and educating the insured people about the benefits of changes in their diets, such as use of low dose aspirin, long term benefits of calcium supplements, use of vitamin D and C, consumption of fruits and vegetables, use of dental floss, use of sunscreen, avoidance of risky behavior, and other lifestyle changes that could be beneficial in improving life spans. For example, wearing seat belts, not driving under the influence of alcohol, and not driving when overly tired are all strategies about which periodic reminders could be helpful.

In some embodiments, all of the major causes of death are examined, determined and then communicated to the insured people on a periodic basis to raise the awareness in the insured people about the relevant risks.

In some related embodiments, the method further includes a step of providing online access to all of the strategies and information outlined above so that customers can see the expected results of any changes that they might make or contemplate making in their lives. Such a web site could facilitate answers to questions that might arise from such customers about their health or life choices and their implications.

Another aspect of the present application provides a method for underwriting life insurances or medical insurances, comprising: (1) screening an insurance candidate for the existence or absence of one or more risk factors, wherein said one or more risk factors comprise nocturia; (2) making a decision on whether to underwrite an insurance policy based on a result of step (1); and (3) if the decision of step (2) is to underwrite an insurance policy, determining an insurance premium based on the result of step (1) and one or more other risk factors in the subject's risk profile.

Another aspect of the present application provides a method for calculating risk associated with nocturia in an insurance candidate/insured subject. The method comprising: providing a computer processor; creating, using the computer processor, a table of correspondence between severity of nocturia and point values associated with the severity of nocturia, wherein the highest point values are assigned to severe nocturia (e.g., >4 voids per night) and lowest point values are assigned to no nocturia (no void per night); assigning, using the table, a corresponding point value to an insurance candidate/insured subject's nocturia severity level; assigning a point value to each of a plurality of other parameters associated with the insurance candidate/insured subject; calculating, using the computer processor, a total point value by summing the corresponding point value for the insurance candidate/insured subject's nocturia severity level and the point value for each of a plurality of other parameters associated with the insurance candidate/insured subject; and determining, using the computer processor, a risk category associated with the insurance candidate/insured subject.

As used herein, the term “processor” refers to computer processors in any general purpose or special purpose computing system environments or configurations. Examples of well-known computing systems, environments, and/or configurations that may be suitable for use with the inventive subject matter include, but are not limited to, personal computers, server computers, hand-held or laptop devices, multiprocessor systems, microprocessor-based systems, set-top boxes, programmable consumer electronics, network PCs, minicomputers, mainframe computers, distributed computing environments that include any of the above systems or devices, and the like. In some embodiments, the processor is a hardware device for executing software that can be stored in a memory. The processor can be, for example, a central processing unit (CPU), data signal processor (DSP) or an auxiliary processor among several processors associated with a server, and a semiconductor based microprocessor (in the form of a microchip) or a macroprocessor.

As used herein, the term “memory” refers to any type of computer storage medium, including long term, short term, or other memory associated with the mobile platform, and is not to be limited to any particular type of memory or number of memories, or type of media upon which memory is stored. The term “memory device” or “data storage device” refers to a physical device that comprises a computer storage medium.

Some portions of this specification are presented in terms of algorithms or symbolic representations of operations on data stored as bits or binary digital signals within a machine memory (e.g., a computer memory). These algorithms or symbolic representations are examples of techniques used by those of ordinary skill in the data processing arts to convey the substance of their work to others skilled in the art. As used herein, an “algorithm” is a self-consistent sequence of operations or similar processing leading to a desired result. In this context, algorithms and operations involve physical manipulation of physical quantities. Typically, but not necessarily, such quantities may take the form of electrical, magnetic, or optical signals capable of being stored, accessed, transferred, combined, compared, or otherwise manipulated by a machine. It is convenient at times, principally for reasons of common usage, to refer to such signals using words such as “data,” “content,” “bits,” “values,” “elements,” “symbols,” “characters,” “terms,” “numbers,” “numerals,” or the like. These words, however, are merely convenient labels and are to be associated with appropriate physical quantities.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

The method of present application may be implemented in the general context of computer-executable instructions, such as program modules, being executed by a computer. Generally, program modules include routines, programs, objects, components, data structures, etc. that perform particular tasks or implement particular abstract data types. The method of present application may also be practiced in distributed computing environments where tasks are performed by remote processing devices that are linked through a communications network. In a distributed computing environment, program modules may be located in both local and remote computer storage media including memory devices.

In some embodiments, the method for managing the cost of life insurances or health insurances, comprises the steps of: (1) screening an insured subject for the presence or absence of one or more risk factors including nocturia, wherein the presence and severity of nocturia is determined by: (a) an answer provided by the insured subject or an authorized representative of insured subject in response to a question about the average frequency of urination at night, and/or (b) measuring a level of urinary prostaglandin E2 (PGE2), urinary prostaglandin F2α (PGF2α), urinary nerve growth factor (UNGF), plasma arginine vasopressin (AVP), plasma atrial natriuretic hormone (ANH), plasma renin-angiotensin aldosterone (RAA) or combinations thereof in the insured subject; (2) providing to the insured subject information about how to reduce risk associated with one or more risk factors present in the insured subject, wherein the information includes information about behavior or dietary exercises that would help to reduce severity of nocturia or to reduce the risks associated with nocturia; and (3) providing incentives to the insured subject to encourage positive behavior by the insured subject, wherein the incentive include reduced insurance premium based on proven performance of one or more positive behavior. In some embodiments, the method further comprises the step of providing the insured subject with a device that would help to encourage the recipient to do physical exercises on a regular basis.

Another aspect of the present application provides a method for screening for insurance candidates. The method includes the steps of (1) generating a risk profile concerning a subject seeking insurance, wherein the risk profile comprises a plurality of risk factors including the severity level of the subject's nocturia; (2) assigning a risk point value to the subject based on the severity level of the subject's nocturia and a risk point matrix; (3) assigning additional risk point values to the subject based on other risk factors in the subject's risk profile; (4) determining a total risk point value of the subject; and (5a) if the total risk point value equals to or exceeds a predetermined threshold value, rejecting the subject as an insurance candidate, or (5b) if the total risk point value is below the predetermined threshold, generating an insurance premium for the subject based on an insurance premium matrix. In some embodiments, the risk point matrix and the insurance premium matrix are stored in a memory device.

Another aspect of the present application provides a method for screening clinical trial candidates. The method comprises the steps of: (1) generating a selection profile concerning a potential candidate for a clinical trial, wherein the selection profile comprises a plurality of selection factors including the severity level of the potential candidate's nocturia; (2) assigning a selection point value to the subject based on the severity level of the potential candidate's nocturia and the risk point matrix; (3) assigning additional selection point values to the subject based on other selection factors in the potential candidate's selection profile; (4) determining a total selection point value of the potential candidate; and (5a) if the total candidate point value equals to or exceeds a predetermined threshold value, accept the potential candidate as a clinical trial candidate, or (5b) if the total risk point value is below the predetermined threshold, reject the potential candidate as a candidate of the clinical trial.

Computer System

Another aspect of the present application relates to a computer system for managing the cost of insurance for a subject. In some embodiments, the computer system comprises a processor and a memory device, wherein the memory device stores a risk point matrix and wherein the processor is configured to: (1) receive a risk profile concerning the subject, wherein the risk profile comprises a plurality of risk factors including the severity level of the subject's nocturia; (2) assign a risk point value to the subject based on the severity level of the subject's nocturia and the risk point matrix stored in the memory device; (3) assign additional risk point values to the subject based on other risk factors in the subject's risk profile; (4) determine a total risk point value of the subject; and (5a) if the total risk point value equals to or exceeds a predetermined threshold value, reject the subject as an insurance candidate, or (5b) if the total risk point value is below the predetermined threshold, generate an insurance premium for the subject based on an insurance premium matrix stored in the memory device. In some embodiments, the processor is further configured to: (6) retrieve information about how to reduce risk associated with nocturia and/or other risk factors from the memory device, and (7) electronically deliver the information to the subject. In some embodiments, the risk factors include age, gender, weight, height, smoking history, job history, use of motor cycles, hang gliding, mountain climbing, or other dangerous pursuits, history of traffic violations, alcohol consumption, drug use history, personal history and family history of cardiovascular disease, personal history and family history of cancer, personal history and family history of immunological disorders, personal history and family history of mental disorders, and personal history and family history of cardiovascular diseases. The insurance can be life insurance or health insurance or long term care insurance or disability insurance. In term of hardware structure, the computer includes, but is not limited to, mainframes, servers, PCs, workstations, laptops, PDAs and the like. In addition to the processor and memory, the computer may include one or more input and/or output (I/O) devices (or peripherals) that are communicatively coupled via a local interface. The local interface can be, but is not limited to, one or more buses or other wired or wireless connections, as is known in the art.

Another aspect of the present application relates to a computer system for managing the cost of insurance for an insured subject. The computer system comprises a processor and a memory, wherein the memory stores a risk point matrix and wherein the processor is configured to: receive a risk profile concerning the subject, wherein the risk profile comprises a plurality of risk factors including the severity level of the subject's nocturia; assign a risk point value to the subject based on the severity level of the subject's nocturia and the risk point matrix stored in the memory; assign additional risk point values to the subject based on other risk factors in the subject's risk profile; and retrieve information about positive behavior that reduces risks associated with nocturia and/or other risk factors from the memory and electronically deliver the information to the subject. The information about positive behavior may comprise, for example, information about the benefits of physical exercise, mental exercise, changes in the diet, use of low dose aspirin, use of calcium supplements, use of vitamin D and C, consumption of fruits and vegetables, use of dental floss, use of sunscreen, avoidance of risky behavior, wearing seat belts, not driving under the influence of alcohol, and not driving when overly tired.

In some embodiments, the processor is further configured to: receive information about confirmed performance of positive behavior; and determine, based on an incentive matrix stored in the memory device, an incentive to encourage continued performance of the positive behavior. The incentive matrix provides various incentives to different levels and/or types of positive behavior, and is modified from time to time. In some embodiments, the processor is further configured to: electronically provide periodic reminders to the insured subject, wherein the periodic reminder comprises information on the benefit of positive behavior.

Another aspect of the present application relates to a computer system for selecting candidate for a clinical trial. The computer system comprises a processor and a memory device, wherein the memory device stores a selection point matrix and wherein the processor is configured to: (1) receive a selection profile concerning a potential candidate, wherein the selection profile comprises a plurality of selection factors including the severity level of the potential candidate's nocturia; (2) assign a selection point value to the subject based on the severity level of the potential candidate's nocturia and the risk point matrix stored in the memory device; (3) assign additional selection point values to the subject based on other selection factors in the potential candidate's selection profile; (4) determine a total selection point value of the potential candidate; and (5a) if the total candidate point value equals to or exceeds a predetermined threshold value, accept the potential candidate as a clinical trial candidate, or (5b) if the total risk point value is below the predetermined threshold, reject the potential candidate as a candidate of the clinical trial. In some embodiments, the processor is further configured to: (6) retrieve information about how to reduce risk associated with nocturia from the memory device, and (7) electronically deliver the information to the potential candidate.

In some embodiment, the processor may operate to support performance of the relevant operations in a “cloud computing” environment or as an SaaS. In some embodiments, at least some of the operations are performed by a group of computers (as examples of machines including processors), these operations being accessible via a network (e.g., the Internet) and via one or more appropriate interfaces (e.g., APIs). In some embodiments, the performance of certain of the operations are distributed among the one or more processors deployed within a single machine, or across a number of machines. In some embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

### Computer Readable Medium

Another aspect of the present application provides a tangible computer readable medium having instructions stored thereon for insurance underwriting based on risk factors including the severity of nocturia. The instructions when executed by a processor configure the processor to: (1) create a table of correspondence between severity of nocturia and point values associated with the severity of nocturia , wherein the highest point values are assigned to very severe nocturia and lowest point values are assigned to no nocturia; (2) assign, using the table, a corresponding point value to an insurance candidate/insured subject's nocturia severity level; (3) assign a point value to each of a plurality of other parameters associated with the insurance candidate/insured subject; (4) calculate, using the computer processor, a total point value by summing the corresponding point value for the insurance candidate/insured subject's nocturia severity level and the point value for each of a plurality of other parameters associated with the insurance candidate; and (5) determine, using the computer processor, a risk category associated with the insurance candidate/insured subject. The insurance may be life insurance, health insurance, long term care insurance or disability insurance.

The tangible computer readable medium includes both volatile and nonvolatile, removable and non-removable media implemented in any method or technology for storage of computer-readable instructions, data structures, program modules, or other data. Examples of tangible computer readable medium include, but are not limited to, RAM, ROM, EEPROM, flash memory, CD-ROM, digital versatile disks (DVD) or other optical disk storage, magnetic cassettes, magnetic tape, magnetic disk storage, or other magnetic storage devices, or any other medium that can be used to store the desired information and that can be accessed by a computer.

In some embodiments, the tangible computer readable medium has instructions stored thereon for insurance underwriting based on risk factors including the severity of nocturia, the instructions when executed by a processor causing the processor to: (1) receive risk profile concerning the subject, wherein the risk profile comprises a plurality of risk factors including the severity level of the subject's nocturia; (2) assign a risk point value to the subject based on the severity level of the subject's nocturia and the risk point matrix stored in the memory device; (3) assign additional risk point values to the subject based on other risk factors in the subject's risk profile; (4) determine a total risk point value of the subject; and (5a) if the total risk point value equals to or exceeds a predetermined threshold value, indicate that the subject is rejected as an insurance candidate, or (5b) if the total risk point value is below the predetermined threshold, generate an insurance premium for the subject based on an insurance premium matrix stored in the memory device.

In some embodiments, the tangible computer readable medium has instructions that, when executed by a processor, cause the processor to: retrieve information about how to reduce risk associated with nocturia and/or other risk factors from the memory device, and electronically deliver the information to the subject. In some embodiments, the risk factors comprise age, gender, weight, height, diet, drinking habit, smoking history, job history, use of motor cycles, hang gliding, mountain climbing, or other dangerous pursuits, history of traffic violations, alcohol consumption, drug use history, personal history and family history of cardiovascular disease, personal history and family history of cancer, personal history and family history of immunological disorders, personal history and family history of mental disorders, and personal history and family history of cardiovascular diseases.

Another aspect of the present application relates to a tangible computer readable medium comprising instructions stored thereon for selecting candidate for a clinical trial based on selection factors including the severity of nocturia. The instructions when executed by a processor causing the processor to: (1) receive a selection profile concerning a potential candidate, wherein the selection profile comprises a plurality of selection factors including the severity level of the potential candidate's nocturia; (2) assign a selection point value to the potential candidate based on the severity level of the potential candidate's nocturia and the risk point matrix stored in a memory; (3) assign additional selection point values to the subject based on other selection factors in the potential candidate's selection profile; (4) determine a total selection point value of the potential candidate; and (5a) if the total candidate point value equals to or exceeds a predetermined threshold value, accept the potential candidate as a clinical trial candidate, or (5b) if the total risk point value is below the predetermined threshold, reject the potential candidate as a candidate of the clinical trial. In some embodiments, a lower selection point value is assigned to a candidate with more severe nocturia. In one embodiment, a selection point value of 3 is assigned to a candidate of no nocturia, a selection point value of 2 is assigned to a candidate of mild nocturia, a selection point value of 1 is assigned to a candidate of moderate nocturia, and a selection point value of 0 is assigned to a candidate of severe nocturia.

In some embodiments, the instructions stored in the memory or tangible computer readable medium may include one or more separate software programs, each of which comprises an ordered listing of executable instructions for implementing logical functions. In some embodiments, the software programs in the memory or tangible computer readable medium include a suitable operating system and a number of functional components that contain executable instructions for the processor to complete the task described above.

In some embodiments, the tangible computer readable medium contains a software with a plurality of modules. Each module contains instructions that, when executed by a processor, cause the processor to perform a certain set of functions. In some embodiments, the software comprises a profile creation module that receives information about a subject and creates a risk profile or candidate profile for the subject, an risk analysis module that determines a total risk point value or total selection point value based on a risk factor/risk point value matrix or a selection factor/selection point value matrix stored in a memory, and makes a determination based on the total risk point value or total selection point value; a matrix maintenance module that updates the risk factor/risk point value matrix or the selection factor/selection point value matrix; and an information module that collects information about risk factors, as well as positive and negative behavior associate with the risk factors, and deliver the information electronically to insured subjects. In some embodiments, the software further comprises an incentive module that processes all incentive-related information and transactions.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter of the present disclosure.

Additionally, certain embodiments are described herein as including logic or a number of components, modules, or mechanisms. Modules may constitute either software modules (e.g., code stored on a machine-readable medium) or hardware modules. A hardware module is tangible unit capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

A hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module in dedicated and permanently configured circuitry or in temporarily configured circuitry (e.g., configured by software) may be driven by cost and time considerations.

Accordingly, the term hardware should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware and software modules can provide information to, and receive information from, other hardware and/or software modules. Accordingly, the described hardware modules may be regarded as being communicatively coupled. Where multiple of such hardware or software modules exist contemporaneously, communications may be achieved through signal transmission (e.g., over appropriate circuits and buses) that connect the hardware or software modules. In embodiments in which multiple hardware modules or software are configured or instantiated at different times, communications between such hardware or software modules may be achieved, for example, through the storage and retrieval of information in memory structures to which the multiple hardware or software modules have access. For example, one hardware or software module may perform an operation and store the output of that operation in a memory device to which it is communicatively coupled. A further hardware or software module may then, at a later time, access the memory device to retrieve and process the stored output. Hardware and software modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

The above description is for the purpose of teaching the person of ordinary skill in the art how to practice the present application, and is not intended to detail all those obvious modifications and variations of it that will become apparent to the skilled worker upon reading the description. It is intended, however, that all such obvious modifications and variations be included within the scope of the present application, which is defined by the following claims. The claims are intended to cover the components and steps in any sequence that is effective to meet the objectives there intended, unless the context specifically indicates the contrary. All of the references and patent disclosures cited in the specification are expressly incorporated by reference in their entirety herein.

What is claimed is:

1. A computer system, comprising a processor and a memory device, wherein the memory device stores a risk point matrix and wherein the processor is configured to:
(1) receive or generate a risk profile concerning the subject, wherein the risk profile comprises a plurality of risk factors including a severity level of the subject's nocturia;
(2) assign a risk point value to the subject based on the severity level of the subject's nocturia and a risk point matrix stored in the memory device;
(3) assign additional risk point values to the subject based on other risk factors in the subject's risk profile;
(4) determine a total risk point value of the subject; and
(5a) if the total risk point value equals to or exceeds a predetermined threshold value, reject the subject as an insurance candidate, or
(5b) if the total risk point value is below the predetermined threshold, generate an insurance premium for the subject based on an insurance premium matrix stored in the memory device.

2. The computer system of claim 1, wherein the processor is further configured to:
(6) retrieve information about how to reduce risks associated with nocturia and/or other risk factors from the memory device, and
(7) electronically deliver the information to the subject.

3. The computer system of claim 1, wherein the risk factors comprise age, gender, weight, height, smoking history, job history, use of motor cycles, hang gliding, mountain climbing, history of traffic violations, alcohol consumption, drug use history, personal history and family history of cardiovascular disease, personal history and family history of cancer, personal history and family history of immunological disorders, personal history and family history of mental disorders, personal history and family history of cardiovascular diseases.

4. The computer system of claim 1, wherein the insurance is life insurance or health insurance or long term care insurance or disability insurance.

5. A tangible computer readable medium having instructions stored thereon for insurance underwriting based on risk factors including the severity of nocturia, the instructions when executed by a processor causing the processor to:
(1) receive or generate a risk profile concerning the subject, wherein the risk profile comprises a plurality of risk factors including the severity level of the subject's nocturia;
(2) assign a risk point value to the subject based on the severity level of the subject's nocturia and the risk point matrix stored in the memory device;
(3) assign additional risk point values to the subject based on other risk factors in the subject's risk profile;
(4) determine a total risk point value of the subject; and
(5a) if the total risk point value equals to or exceeds a predetermined threshold value, reject the subject as an insurance candidate, or
(5b) if the total risk point value is below the predetermined threshold, generate an insurance premium for the subject based on an insurance premium matrix stored in the memory device.

6. The computer readable medium of claim 5, wherein the instructions when executed by a processor further causing the processor to:
(6) retrieve information about how to reduce risk associated with nocturia and/or other risk factors from the memory device, and
(7) deliver the information to the subject.

7. The computer readable medium of claim 5, wherein the risk factors comprise age, gender, weight, height, smoking history, job history, use of motor cycles, hang gliding, mountain climbing, history of traffic violations, alcohol consumption, drug use history, personal history and family history of cardiovascular disease, personal history and family history of cancer, personal history and family history of immunological disorders, personal history and family history of mental disorders, and personal history and family history of cardiovascular diseases.

8. The computer readable medium of claim 5, wherein the insurance is life insurance or health insurance or long term care insurance or disability insurance.

9. A method for managing the cost of life insurances or health insurances, comprising:
screening an insured subject for the presence or absence of one or more risk factors including nocturia, wherein the presence and severity of nocturia is determined by:
(1) an answer provided by the insured subject or an authorized representative of insured subject or medical professional in response to a question about the average frequency of urination at night, and/or
(2) measuring a level of urinary prostaglandin E2 (PGE2), urinary prostaglandin F2α (PGF2α), urinary nerve growth factor (UNGF), plasma arginine vasopressin (AVP), plasma atrial natriuretic hormone (ANH), plasma renin-angiotensin aldosterone (RAA) or combinations thereof in the insured subject;
providing to the insured subject information about how to reduce risk associated with one or more risk factors present in the insured subject, wherein the information includes information about behavior, or medications, or diet, or drinking habit, or exercises that would help to reduce severity of nocturia or to reduce the risks associated with nocturia; and
providing incentives to the insured subject to encourage positive behavior by the insured subject, wherein the incentive include reduced insurance premium or discounts or other form of financial or other reward based on proven performance of one or more positive behavior.

10. The method of claim 9, further comprising the step of providing the insured subject with, or an incentive to obtain, (1) a device that would help to encourage the recipient to do physical exercises on a regular basis or (2) a device that monitors the subject's health or stress levels and sends out an alert if evidence of increasing risk or need for medical care becomes apparent.

11. A computer system, comprising a processor and a memory device, wherein the memory device stores a risk point matrix and wherein the processor is configured to:
Receive or generate a risk profile concerning the subject, wherein the risk profile comprises a plurality of risk factors including the severity level of the subject's nocturia;
assign a risk point value to the subject based on the severity level of the subject's nocturia and the risk point matrix stored in the memory device;
assign additional risk point values to the subject based on other risk factors in the subject's risk profile; and
retrieve information about positive behavior that reduces risks associated with nocturia and/or other risk factors from the memory device and deliver the information to the subject.

12. The computer system of claim 11, wherein the information about positive behavior comprises information about the benefits of physical exercise, mental exercise, changes in the diet, use of low dose aspirin, use of calcium supplements, use of vitamin D and C, consumption of fruits and vegetables, use of dental floss, use of sunscreen, avoidance of risky behavior, wearing seat belts, not driving under the influence of alcohol, and not driving when overly tired.

13. The computer system of claim 11, wherein the processor is further configured to:
receive information about confirmed performance of positive behavior; and
determine, based on an incentive matrix stored in the memory device, an incentive to encourage continued performance of the positive behavior.

14. The computer system of claim 11, wherein the processor is further configured to:
electronically provide periodic reminders to the insured subject, wherein the periodic reminder comprises information on the benefit of positive behavior.

15. The computer system of claim 11, wherein the information about positive behavior that reduces risks associated with nocturia and/or other risk factors from the memory device is delivered directly to the subject electronically by the computer system.

16. A computer system, comprising a processor and a memory device, wherein the memory device stores a selection point matrix and wherein the processor is configured to:
(1) receive or generate a selection profile concerning a potential candidate, wherein the selection profile comprises a plurality of selection factors including the severity level of the potential candidate's nocturia;
(2) assign a selection point value to the subject based on the severity level of the potential candidate's nocturia and the risk point matrix stored in the memory device;
(3) assign additional selection point values to the subject based on other selection factors in the potential candidate's selection profile;
(4) determine a total selection point value of the potential candidate; and
(5a) if the total selection point value equals to or exceeds a predetermined threshold value, accept the potential candidate as a clinical trial candidate, or
(5b) if the total selection point value is below the predetermined threshold, reject the potential candidate as a candidate of the clinical trial.

17. The computer system of claim 16, wherein the processor is further configured to:
(6) retrieve information about how to reduce risk associated with nocturia from the memory device, and
(7) deliver the information to the potential candidate.

18. The computer system of claim 17, wherein the information in step (7) is delivered directly to the potential candidate electronically by the computer system.

19. A tangible computer readable medium comprising instructions stored thereon for selecting candidate for a clinical trial based on selection factors including the severity of nocturia, the instructions when executed by a processor causing the processor to:
(1) receive or generate a selection profile concerning a potential candidate, wherein the selection profile comprises a plurality of selection factors including the severity level of the potential candidate's nocturia;
(2) assign a selection point value to the potential candidate based on the severity level of the potential candidate's nocturia and the risk point matrix stored in the memory device;
(3) assign additional selection point values to the subject based on other selection factors in the potential candidate's selection profile;
(4) determine a total selection point value of the potential candidate; and
(5a) if the total selection point value equals to or exceeds a predetermined threshold value, accept the potential candidate as a clinical trial candidate, or
(5b) if the total selection point value is below the predetermined threshold, reject the potential candidate as a candidate of the clinical trial.
